(19) United States
(12) Patent Application Publication
(10) Pub. No.: US 2010/0160268 A1
(43) Pub. Date: Jun. 24, 2010

(54) Isoflavonoid Analogs and their Metal Conjugates as Anti-Cancer Agents

(76) Inventors: Sarkar Fazlul, Plymouth, MI (US); Subhash Padhye, Pune (IN)

Correspondence Address: Rohm & Monsanto, PLC, 12 Rathbone Place, Grosse Pointe, MI, 48230, US

(21) Appl. No.: 11/992,458

(22) PCT Filed: Sep. 25, 2006
(86) PCT No.: PCT/US06/37299

§ 371 Date: Mar. 21, 2008

Related U.S. Application Data
(60) Provisional application No. 60/720,358, filed on Sep. 23, 2005.

(30) Foreign Application Priority Data
Jun. 2, 2006 (US) 11/445929

(51) Int. Cl. A61K 31/57; C07J 51/00; C07J 41/00; C07F 1/08; C07D 311/22; A61K 31/555; A61K 31/352; A61P 35/00; C40B 40/04

(52) U.S. Cl. 514/169; 552/504; 552/517; 549/210; 549/401; 514/186; 514/456; 506/15

(57) ABSTRACT

A pharmacologic agent for treating and/or preventing cancer, among other diseases and conditions, and particularly breast, prostate, and pancreatic cancer, in humans and animals. The novel pharmacologic agent is an isoflavonoid or isoflavonoid mimetic covalently attached to a cytotoxic pharmacophore that, preferably has the ability to conjugate with a metal salt to form a more potent metal complex, particularly a Cu(II) complex. The isoflavonoid or isoflavonoid mimetic may he non-fragmented steroidal hormone, such as progesterone which is structurally related to the isoflavone genistein, or a small molecule hormone mimetic, such as chromone. An illustrative non-fragmented steroidal embodiment is 17-acetyl-10,13-dimethyl-1,2,6,7,8,9,11,12,13,14,15, 16,17-tetradecahydrocyclopenta[a]phenanthren-3-thiosemicarbazone and its Cu(II) complex. Effective chromone analogs include the thiosemicarbazone and hydrazone analogs of 4-oxo-4H-chromene-3-carboxaldehyde and their Cu(IT) complexes.

### RELATIONSHIP TO OTHER APPLICATION(S)

This application claims the benefit under 35 U.S.C. §119 of U.S. Provisional Patent Application Ser. No. 60/720,358 filed on Sep. 23, 2005 and U.S. Ser. No. 11/445,929 filed on Jun. 2, 2006, the texts of which are incorporated herein by reference.

### BACKGROUND OF THE INVENTION

1. Field of the Invention

This invention relates generally to novel analogs of isoflavone and metal complexes thereof, and more particularly to isoflavonoid or isoflavonoid mimetics that are useful for preventing and/or treating diseases, such as cancer.

2. Description of the Related Art

The lower incidence of breast and prostate cancer among Asians, who consume 20-50 times more soy than Americans, has raised the question as to whether soy in the diet acts as a natural chemoprotective agent. Isoflavones in soy, including genistein, daidzein, glycitein, and others, are the active agents in this regard. However, genistein (4,5,7,-trihydroxyisoflavone) has been demonstrated to be the principal isoflavone in soy responsible for reducing the incidence of hormone-related cancers. Other dietary agents, such as indole-3-carbinol, curcumin, resveratrol, and green or black tea polyphenols, have also been shown to be capable of killing cancer cells in vitro and to have anti-tumor activity against multiple types of cancers in in vivo animal studies.

Because of its structural similarity to 17β-estradiol, genistein, which is also known as a phytoestrogen, has been shown to compete with 17β-estradiol for estrogen receptor binding resulting in agonistic or antagonistic activity. Studies have demonstrated that genistein causes inhibition of cell growth in various cancer cell lines, including breast and prostate cancers, in vivo and in vitro. Additional studies have confirmed that genistein exerts an inhibitory effect on the development of cancers, cancer cell growth, and cancer progression, as well as cancer cell invasion, metastasis, and angiogenesis. From gene expression profiles, genistein has been found to regulate the genes that are critical for the control of cell proliferation, cell cycle, apoptosis, oncogenesis, transcription regulation, and cell signal transduction pathways. These results suggest that genistein is a promising agent for cancer prevention and/or treatment. For a review article on the molecular mechanisms of action of genistein, including the effects of genistein on cell cycle, apoptosis, estrogen receptor, androgen receptor, NF-κB, Akt, and MAPK pathways, see Sarkar, et al., The Role of Genistein and Synthetic Derivatives of Isoflavone in Cancer Prevention and Therapy, Mini-Reviews in Medicinal Chemistry, Vol. 6, No. 4, pages 401-407 (2006), the disclosure of which is incorporated by reference.

It is clear from the reported research that genistein causes a pleiotropic effect on cancer cells. However, genistein alone may not be potent enough to treat and/or prevent cancers. There is, therefore, a need for synthetic analogs or derivatives of the isoflavone genistein that have more robust biological properties.

Referring to FIG. 11, the basic structural feature of genistein is the flavone nucleus which is composed of two benzene rings (A and B) linked through a heterocyclic pyrane ring (C). Because of the structural similarity to estrogen (17β-estradiol), genistein, is capable of influencing and modulating the action of estrogen. However, genistein also exerts its own biological effects that are distinct from estrogen. Since biological activity is related to molecular structure, changes in molecular structure have been shown to cause extensive changes in biological activity. Therefore, derivatives of genistein (and consequently hormone mimetics) based on the structural motifs of genistein, and other naturally-derived known phytochemicals, may have greater ability to prevent and/or treat cancers than the natural products themselves. Certainly, synthetic derivatives of these natural phytochemicals maybe easier to produce on a commercial scale.

Referring now to FIG. 1, it is evident that the assembly of blocks (1-3) that can be used to build the steroidal scaffold of progesterone (4), for example, are themselves distinct chemical entities which are formed by biosynthetic pathways in both plants and animals. Many of these chemical entities are consumed by humans in nutritional sources which help provide the physiologically active constituents needed for maintenance of life and health. Except for a few nutrients, many of these dietary chemicals have remained uncharacterized. Some of these chemicals are inert, some are toxic or carcinogenic, while others may have positive effects on physiologic function acting as protective agents countering the risk of acute toxicity and diminishing the onset of chronic diseases including cancer.

Referring again to FIG. 1, the steroidal motif found in isoflavone, is a self-assembling framework consisting of a variety of chemical structures that serve as the building blocks for creating a matrix, of steroidal and non-steroidal compounds of therapeutic and nutritional importance. Each column of this matrix can be expanded by derivatizing the basic scaffold with additional substituents and pharmacophores, finally yielding an analog of a naturally-occurring phytochemical that can be used clinically for the prevention and/or treatment, or as adjuvant to a treatment, of many chronic disorders, including cancer. Of the many possible building blocks of the steroidal motif, genistein (5) is outstanding due to its well-proven biological activities and influences as described hereinabove. These same structural motifs are present in other naturally-occurring dietary agents, such as indole-3-carbinol (cruciform vegetables), resveratrol (red wine), curcumin (curry), green and black tea polyphenols, etc. as shown on FIG. 1.

There is, thus, a need for synthetic analogs or derivatives of isoflavone and other naturally-occurring dietary agents that are more potent than the naturally-occurring product and that can be synthesized on a commercial scale.

Of course, there is also a need for a method of rapidly screening the many possible combinations of small molecule analogs of these naturally-occurring compounds for efficacy and toxicity.

### SUMMARY OF THE INVENTION

The foregoing and other objects are addressed by this invention which provides active pharmacologic agents for treating and/or preventing cancer, among other diseases and conditions, and particularly breast, prostate, and pancreatic cancer, in humans and animals. The active pharmacologic agents of the present invention selectively target receptors of the type over-expressed in malignant cells and comprise ligands of a cytotoxic pharmacophore covalently attached to a carrier. The carrier may be an isoflavonoid or an isoflavonoid mimetic. As used herein, the term “isoflavonoid mimetic” refers to a molecule that has a steroidal motif derived from isoflavone, and in particularly preferred embodiments, from the isoflavone genistein. The isoflavonoid mimetic may be, in some embodiments, a non-fragmented steroidal hormone, such as progesterone or estrogen, or in other embodiments, a small molecule analog of isoflavone, such as 3-formylchromone. The ligand is preferably conjugated to a transition metal ion. The resulting metal complexes have an overall lipophilic nature that is greater than the parent ligand from which they are derived and therefore, their cellular internalization is improved.

In a first embodiment, the pharmacologic agent comprises a carrier that is a non-fragmented steroidal hormone, such as estrogen, progesterone, testosterone, hydrocortisone or prednisone which is appended to a cytotoxic pharmacophore that is, preferably, capable of forming a metal complex. In a specific illustrative embodiment, described in detail below, the non-fragmented hormonal molecule is progesterone.

The pharmacophores have been chosen for their ability to conjugate with metals, such as transition metal ions that may have affinities for steroidal receptors (and others), as well as for their known ability to be cytotoxic to cancer cells; or to otherwise exert biological effects on signal transduction intermediates such as epidermal growth factor receptor (EGFR), Akt, or NF-κB, or on the enzymes obligatory for DNA synthesis.

Exemplary pharmcophores include, without limitation, amines, alkylamines, arylamines, heterocyclic amines, phenylamines, naphthoylamines, isothiocyanates, semicarbazides, thiosemicarbazides, hydrazones, thiourea, hydroxamates, arylazo, azocylic, carboxyamidrazones, ferrocenes and substituted ferrocenes.

In the specific embodiments presented herein, the pharmacophores are thiosemicarbazone, benzoyl hydrazide, isonicotinoyl hydrazide, and salicylic hydrazide.

As indicated above, particularly preferred compounds are metal complexes (1:1 ligand to metal stoichiometry) of transition metal ions, such as vanadium, chromium, manganese, iron, cobalt, nickel, copper, molybdenum, ruthenium, platinum, palladium and zinc. Preferred metals are copper, nickel, and platinum, which are particularly known for their therapeutic effects. Most preferred, however, is copper (II) which has two distinct advantages over other metals, including 2 0 platinum, specifically its easily tunable redox potential through appropriate ligand framework with its potential intrinsic affinity for the estrogen receptor. There was an inverse relationship between IC₅₀ values and the half-wave potentials of Cu⁺²/Cu⁺¹ redox couples for these compounds. In view of this, metal redox potentials may be a useful criteria in the design of metal-based anti-cancer agents. Positive metal redox potential allows reversible conversions into cuprous and cupric species which are linked to the conversions of intracellular molecular oxygen into superoxide anions and subsequent hydrogen peroxide which can trigger apoptosis.

Metal conjugates of the ligands of the present invention were found to exhibit synergistic enhancement in their antiproliferative activities with, in preferred embodiments, IC₅₀ values around ≦5 μM. Thus, these compounds are highly likely to achieve effectiveness under in vivo conditions. A synergistic effect was also found in the pro-apoptotic activity of the metal conjugates.

In preferred embodiments, the carrier is progesterone. The progesterone motif is similar to the isoflavone, genistein. A particularly preferred embodiment, is the thiosemicarbazone and its Cu(II) complex: 17-acetyl-10,13-dimethyl-1,2,6,7,8,9,11,12,13,14,15,16,17-tetradecahydrocyclopenta[a]phenanthren-3-thiosemicarbazone.

In a second embodiment, non-steroidal pharmacologic agents are provided that are isoflavonoid mimetics based on a smaller molecule that is an analog of a naturally-occurring molecule, such as the isoflavone genistein. In a particularly preferred embodiment, the isoflavonoid mimetic is chromone. The structure-activity correlations for the isoflavonoid compounds have indicated that certain features, desirable for the anti-tumor properties of these compound include a benzopyran motif, with a double bond between the C2-C3 positions, and a side chain containing a phenyl ring having metal-2 chelating ability. These features can be built into 3-formylchromone by condensing it with various amines in an alcoholic medium to form a Schiff base ligand. The Schiff base chelates easily with a salt of a transition metal to form a conjugate with potent radical scavenging properties.

Preferred derivatives are analogs of the flavonoid genistein, and in particular, the thiosemicarbazone and hydrazone analogs of 4-oxo-4H-chromene-3-carboxaldehyde (herein designated as chromone)and their Cu(II) complexes. Specific preferred embodiments include:

4-ox o-4H-chromene-3-carboxaldehyde-thiosemicarbazone;

4-oxo-4H-chromenc-3-carboxaldehyde-benzoylhydrazone;

4-oxo-4H-chromene-3-carboxaldehyde-isonicotinoylhydrazone; and

4-oxo-4H-chromene-3-carboxaldehyde-salicylhydrazone.

Referring to FIG. 18, a generic chemical structure for the preferred embodiments of the invention is shown.

In a further composition of matter aspect of the invention, a formulation comprises a therapeutically effective amount of an active pharmacologic agent(s) in accordance with the present invention in a delivery vehicle. The pharmacologic agent may be the free drug or a pharmaceutically acceptable salt thereof. The term pharmaceutically acceptable salt includes, at least, the commonly used alkali metal salts used to form addition salts of free acids or free bases.

It is contemplated that the pharmacologic agents of the present invention can be formulated for delivery in any route of administration. For oral administration, the pharmacologic agent(s) can be delivered dry in the form of a tablet or capsule, or as a liquid solution or suspension. Oral drug delivery forms are well-known and typically include, conventional additives, such as binders and fillers, disintegrants, lubricants, and the like. For intravenous, intramuscular, subcutaneous, or intraperitoneal administration, the active pharmacologic agent may be combined with a sterile aqueous solution, such as saline or dextrose, preferably isotonic. Of course, a liquid injectable formulation can include other components, such as excipients, anti-oxidants, buffers, osmolarity adjusting agents, and the like, as are known in the art. In addition to conventional drug delivery approaches, it is within the contemplation of the invention that the pharmacologic agents of the present invention can be administered in targeted delivery media, such as in microparticle and nanoparticle formulations.

The pharmacologic agents of the present invention can be used alone, or in combination, with other therapeutic agents, including anti-cancer agents, such as cisplatin or gemeitabine. The pharmacologic agents can be used as an adjuvant, before, after, or concurrent with the administration of other medications or treatment therapies, such as radiation therapy.

In a method of treating aspect of the invention, a therapeutically effective amount of an active pharmacologic agent in accordance with the invention is administered to a patient as a preventative or therapeutic for a disease that is targeted by the active pharmacologic agent. In a preferred exemplary embodiment, cancer, such as breast, prostate, and pancreatic cancer, is treated by selectively targeting steroidal hormone receptors of the type over-expressed in malignant cells with the pharmacologic agents of the present invention.

As used herein, the term “therapeutically-effective” refers to an amount of pharmacologic agent that produces an ameliorating effect in the treatment and/or prevention of cancer, or other targeted disease, and is not toxic to the patient, and preferably does not produce excessive adverse side effects. In a method of making embodiment, a simple synthetic protocol is provided with optimal yield and simple purification. In an illustrative embodiment of this aspect of the invention, a ligand comprising the Schiff base of the desired carrier moiety is synthesized by condensing equimolar amounts of the carrier and various amines. The ligand is combined, in a stochiometric ratio, with a solution of a salt the desired transition metal, which in specific illustrative embodiments, may be halides of copper, nickel, or platinum. The metal conjugate precipitates from solution and is easily purified by standard chromatographic work-up

The molecules, once conjugated with copper, for example, have an E-tautomeric arrangement (square planar geometry). With respect to the geometrical isomers, the cis or trans isomers are based on the placement of the two chloro groups around the copper ion. The cis isomer is preferred, and is preferably formed by employing a sulfur-nitrogen bidentate ligand in the synthetic technique described herein.

In another aspect of the invention, the steroidal scaffold of the hormones (e.g., estrogen, progesterone, or testosterone) are broken down into privileged core structures of known natural products, such as flavonoids, indoles and others, specifically including known phytochemicals, and appended to appropriate pharmacophores capable of conjugating with therapeutically important metal ions. Preferably, the pharmacophores will also have cytotoxic effects on the targeted cancer cells. The privileged core structures and the pharmacophores result in a library of small molecular weight compounds, specifically small flavonoid-like molecules, based on genistein, and their metal conjugates. The compounds of the library of FIG. 2 are specifically within the contemplation of the present invention. The specific illustrative embodiments, described in detail herein, are shown in the matrix of FIG. 2, specifically columns 01 to 03 are progesterone, genistein and 3-formyl chromone, respectively. In this particular example, there are seven rows of known pharmacophores (SC1 to SC7), that may be appended to the scaffold structure (01 to 11) listed in the columns. These pharmacophores, include the pharmacophores used in the specific illustrative embodiments described hereinbelow: thiosemicarbazole (SC1), benzoyl hydrazide (SC3), isonicotinoyl hydrazide (SC4), and salicylic hydrazide (SC6). The resulting compounds are then coordinated with metal ions (not shown in this figure). Typically, the pharmacophores are covalently attached to the carrier scaffold at the equivalent of benzene ring A in the isoflavone motif (see, FIG. 11; Formula I on FIG. 18).

Each of these compounds was tested in a cellular assay to determine its ability to inhibit proliferation of specific cancer cell lines based on concentration and time dependence. Compounds showing consistent results and lowest IC₅₀ values, preferably in the range of nanomols to 10 μM, and preferably less than 5 μM, were considered suitable for further studies in vivo using validated animal models. Compounds having an IC₅₀ value of greater than 20 μM were considered to be not useful. Molecules showing statistically valid responses, without apparent toxicities to animals, are considered as lead compounds. The promising compounds are structurally characterized by a variety of physical methods including elemental analyses, spectroscopy, magnetism, EPR, NMR, cyclic voltammetry and single crystal x-ray diffraction studies. This matrix has provided lead compounds capable of killing tumor cells efficiently and selectively, especially in breast, prostate and pancreatic cancers.

By robust molecular modeling together with computational chemistry, a virtual library can be produced to assist in predicting which molecules should be synthesized and tested for biological activity. The modeled compounds are docked in a receptor and examined for favorable interactions with crucial amino acid residues on the receptor proteins. Preferably, docking studies are performed in a variety of receptors. Those compounds that show promise are then synthesized, and preferably conjugated with a metal ion. The synthesized structures can be tested for biological activity in vitro and in vivo. This enables rapid development of highly effective pharmacologic agents. For compounds already synthesized, modeling corroborates the biological data.

### BRIEF DESCRIPTION OF THE DRAWING

Comprehension of the invention is facilitated by reading the following detailed description, in conjunction with the annexed drawing, in which:

FIG. 1 is a schematic representation of the chemical structure of the building blocks of the steroidal motif illustrating how derivitization of the basic building blocks of progesterone, in this example, results in small molecule analogs of naturally-occurring bioactive phytochemicals;

FIG. 2 is an illustrative matrix of compounds consisting of carriers and pharmacophores comprising a library of useful structures based on analogs of flavonoids;

FIG. 3 is a schematic representation of a chemical reaction scheme for synthesizing a steroidal embodiment of a pharmacologic agent in accordance with the invention and its metal conjugate;

FIG. 4 is a cyclic voltammogram of a metallic conjugate of the pharmacologic agent produced according to the reaction scheme of FIG. 3;

FIG. 5 is a computer model of a compound (17-acetyl-10,13-dimethyl-1,2,6,7,8,9,11,12,13,14,15,16,17-tetradecahydrocyclopenta[a]phenanthren-3-thiosemicarbazone; FPA-101) in accordance with the present invention docked onto the ER-α binding site showing extensive hydrogen bonding interactions with GLU 353 and PHE 404 of the main chain as well as some pi interactions with PHE 404;

FIG. 6 is a computer model showing superimposition of docked model structures of FPA-101 (colored purple) and the agonist estradiol (colored yellow) revealing that the side chain in FPA-101 modifies the position of the helix 12 of the estrogen receptor leading to inhibition of co-activator recruitment resulting in enhanced anti-proliferative activity;

FIG. 7(a) is a confocal microscopic image of a control breast cancer cell line (T47D) intact cell membrane with uptake of Hoechst (blue) dye; FIG. 7(b) is an image of the T47D cells following incubation with 10 μM concentration of FPA-102 (the Cu(II) complex of FPA-101) for 72 hours showing cells undergoing cell death by uptake of propidium iodide (red);

FIGS. 7(c) and (d) are graphical plots of an ELISA apoptosis assay in BT20 (c) and PC3 (d) cells in the presence of FPA-102 (control, 10 μM, 20 μM) for 24, 48 and 72 hours;

FIG. 8 is a Western blot of Akt, p-Akt and PARP in PC3 cells treated with FPA-102 for 72 hours;

FIG. 9 is a graphical representation of vascular endothelial growth factor (VEGF) levels in pg/mg after 72 hours of exposure to FPA-102 at several concentration levels;

FIG. 10(a) is a schematic flow chart of a protocol for orthotopic tumor induction in mice and a treatment schedule using a pharmacologic agent of the present invention, FPA-102 (25 mg/kg wt);

FIG. 10(b) is a graphical representation of the body weight and tumor burden in control and treated mice;

FIG. 10(c) is a confocal microscopic image of representative histological features of tumors harvested on sacrifice from control and treated mice wherein the treated tumors show marked necrosis and apoptosis;

FIG. 10(d) are developed EMSA gels showing the NF-κB in two tumor samples collected from control and FPA-102 treated mice. Specificity of NF-κB band is confirmed by the supershift as indicated in this figure.

FIG. 11 is a schematic representation of a chemical reaction scheme for synthesizing a non-steroidal embodiment of a pharmacologic agent in accordance with the invention and its metal conjugate;

FIG. 12 is a computer-generated model of genistein docked into the kinase domain of PKB (Akt) protein;

FIG. 13 is a computer-generated model of the interactions of metal conjugate FPA-124 with the active site of the kinase domain of PKB protein;

FIGS. 14(a)-14(d) are graphical representations, of the inhibitory effects of the copper complexes of 4-oxo-4H-chromene-3-carboxaldehyde-thiosemicarbazone (FPA-124);4-oxo-4H-chromene-3-carboxaldehyde-isonicotinoylhydrazone (FPA-125) and 4-oxo-4H-chromene-3-carboxaldehyde-salicylhydrazone (FPA-127) to the relative growth of cancer cells Colo357, BxPC3, BT20, and PC3, respectively, as a function of concentration in μM;

FIGS. 15(c) and (d) are graphical plots of an ELISA apoptosis assay in genistein, Colo357, BxPC3, BT20, and PC3 cells, respectively, in the presence of metal conjugates FPA-124, FPA-125, and FPA-127 and a control;

FIGS. 16(a) and (b) are graphical representations of the IC₅₀ values (μM) of Compounds FPA-124 to FPA-127 plotted against the metal redox couple, E_{1/2} (V) where the symbol □ is FPA-124, ⋄ is FPA-125, and Δ is FPA-127;

FIGS. 17(a)-(c) present data relating to in vivo experiments in mice in an orthotopic pancreatic tumor model wherein FIG. 17(a) is a graphical representation of the relative body weight of animals treated with a control versus a pharmacologic agent in accordance with the present invention (FPA-124), this being indicative of toxicity; FIG. 17(b) is a graphical representation of relative pancreas weight of primary pancreatic tumors in treated mice versus control mice; and FIG. 17(c) is a gel shift assay showing down-regulation of NF-κB DNA binding activity in primary pancreatic tumors from two representative mice in the treated and control groups; and

FIG. 18 is a generic chemical structure for pharmacologic agents, specifically the ligands, in accordance with the present invention.

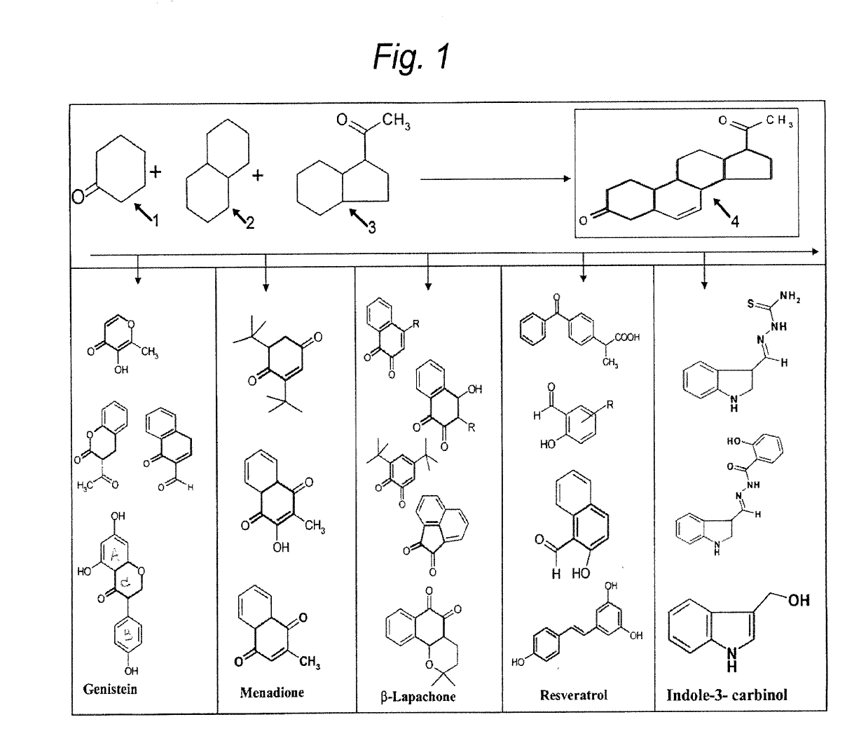

### DETAILED DESCRIPTION

### I. Steroidal Embodiment

### Synthesis of Progesterone Thiosemicarbazone Schiff Base (Compound FPA-101)

Synthesis of Thiosemicarbazide Hydrochloride

Thiosemicarbazide hydrochloride was prepared by adding 4 ml of concentrated hydrochloric acid to a slurry of 4.4 g of powdered thiosemicarbazides in 18 ml of ethanol. The mixture was stirred overnight and the white product was isolated by filtration after washes with cold ethanol to remove excess acid. The product was dried over anhydrous CaCl₂.

Synthesis of Schiff Base

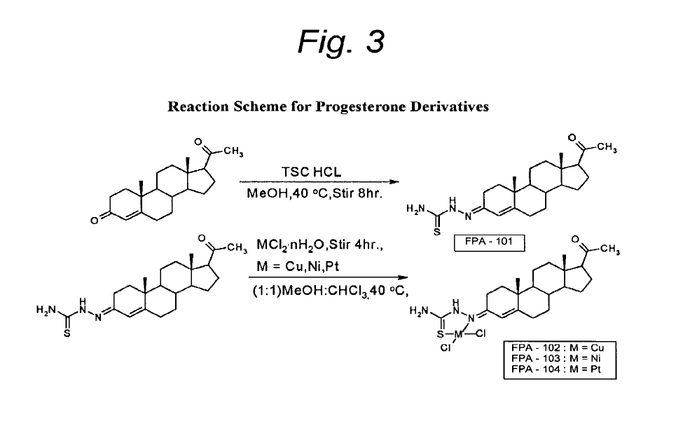

FIG. 3 is an illustrative reaction scheme for producing a Schiff base analog of progesterone, specifically 17-acetyl-10,13-dimethyl-1,2,6,7,8,9,11,12,13,14,15,16,17-tetradecahydrocyclopenta[a]phenanthren-3-thiosemicarbazone (hereinafter designated Compound FPA-101).

An aqueous solution of thiosemicarbazides hydrochloride (0.39 g) and a metabolic solution of progesterone acetate (available commercially from Sigma Chemicals, St. Louis, Mo.; 1 g) were mixed together and the resulting mixture was maintained at 40° C. on a water bath with constant stirring for 8 hours during which the color of the mixture changed from colorless to an intense yellow.

Completion of the reaction was followed by silica gel TLC with chloroform-methanol as the developing solvent, after which the solvent was removed on a rotovapor to obtain a microcrystalline yellow-colored compound. The compound was washed with cold water and dried in a vacuum over anhydrous CaCl₂ (70% yield; Compound FPA-101)

Synthesis of Metal Conjugates (Compounds FPA-102 to FPA-104):

Stoichiometric amounts of the Schiff base Ligand Compound FPA-101 and halides of copper, nickel, or platinum were mixed together in methanol:chloroform solvent (1:1) and stirred at 40° C. for four hours resulting in brown-colored complexes. The reaction mixture was filtered, washed with cold acetonitrile solvent, and dried in a vacuum over anhydrous CaCl₂ (60% yield).

Compositional Studies of FPA-101to FPA-104

The interaction of the progesterone Schiff base FPA-101 with respective metal salts yields metal conjugates having 1:1 stoichiometry with compositional analyses confirming a common molecular formula, viz. [M (ligand) Cl₂]. The non-conducting nature of these metal complexes is revealed from the weak conductivities (3-20 Ωcm² mol⁻¹) observed for them in DMSO and acetonitrile solvents. The copper compound was found to be paramagnetic with a magnetic moment of 1.85 BM while nickel and platinum complexes were diamagnetic suggestive of the square planar geometries for these compounds.

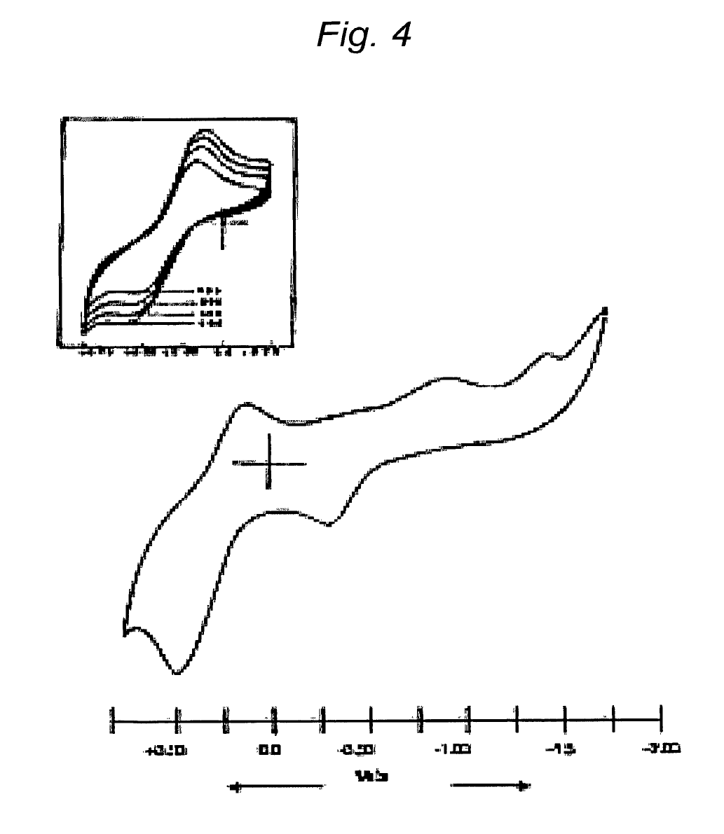

The cyclic voltammographic profiles of FPA-101 and its metal complexes FPA-102 to FPA-104 were recorded in acetonitrile solvent with reference to SCE and using 0.1M TEAP as the supporting electrolyte. The cyclic voltammographic profile of the ligand, shown in FIG. 4, exhibits a broad, irreversible peak at −1.05 V with no anodic counterpart which can be attributed to the reduction of the azomethine chromophore. On metal complexation this peak was shifted to a more positive potential with the appearance of additional irreversible peaks which were ligand-based. Referring to FIG. 4, the only reversible peak centered in the voltammogram of the copper complex was due to Cu²⁺/Cu⁺¹ redox couple centered at +0.47 V. No metal-based peaks were observed for the nickel and platinum compounds.

Physico-Chemical Measurements of FPA-101 to FPA-104

Elemental analyses were carried out in the Microanalytical Lab of University of Pune. The magnetic susceptibilities of the metal complexes were measured at 300 K on Faraday type magnetic balance having field strength of 7000 Gauss. The molecular susceptibilities were corrected for diamagnetism of the component atoms using Pascal's constants. Infrared spectra of the ligand and its metal complexes were recorded as nujol mulls in the range 4500-450 cm⁻¹ on a Perkin Elmer FTIR 283-B instrument while UV-VIS spectra were measured on Genesys-2 machine using 10 mm matched quartz cells. The electrochemical measurements were made in DMF solvent using tetraethylammonium perchlorate (TEAP) as the supporting electrolyte with the help of BAS cyclic voltammetric automatic system CV-27 under dry nitrogen atmosphere. The three-electrode system employed consisted of platinum working electrode, platinum wire as auxiliary electrode and SCE as the reference electrode.

Spectroscopy

The IR spectrum of the Schiff base ligand FPA-101 exhibits strong absorption bands at 3220 and 3120 cm⁻¹ due to asymmetric and symmetric stretching modes of the terminal NH₂ group in the thiosemicarbazone side chain, which are practically unaffected upon metal complexation. A shoulder absorption around 3380 cm⁻¹ is due to the protonated hydrazinic NH group suggesting that the ligand is coordinating to the metal as a neutral moiety. In the mid-IR region the strong absorptions at 1625 and 1590cm⁻¹ are due to the C═O (C-3 carbonyl) and C═C (ring A) stretches. Upon metal complexation a broad band appears around 1610 cm⁻¹ which could be due to displacement of the former absorption resulting in its overlap with the latter. The absorptions due to v N—N stretch (1060 cm⁻¹) and the thiocarbonyl stretch (850 cm⁻¹) are also found to be affected upon complexation indicating their involvement in metal coordination. The assignment of the C═S absorption was difficult due to its mixing with other frequencies over a wide range. However, on the basis of intensity reduction, the absorption at 865 cm⁻¹ was probably the purest v(C═S) vibration. Taken together the IR data confirms the bidentate nature of the Schiff base ligand.

In the electronic spectra of these metal conjugates d-d bands were observed only for the copper complex in DMSO solvent at 15500 and 19200 cm⁻¹ which can be assigned to d_{xy}→>d_{z2} and d_{xy}→d_{xz} transitions in the square planar environment. The bands corresponding to LMCT processes were observed at 22500, 23400 and 25000 cm⁻¹ respectively in this compound.

Electronic Spectra and ESR Spectra of FPA-102

The electronic spectra of the complexes in DMSO solution exhibit characteristic absorption curves corresponding to the square planar geometry. These complexes with dₓ²−y² ground state display three spin-allowed transitions, viz. ²B_{1g}→²A_{2g}(dₓ²−-_{y}²→d_{z}²), ²B_{1g}=²B_{2g}(dₓ²−_{y}²→d_{xy}) and 2B_{1g}→²E_{g}(dₓ²−_{y}²→(d_{xz,yz}) respectively. It is difficult to resolve these bands into individual components due to the closeness of their energies. For Compound FPA-102, the first transition appears as a shoulder at 17240 cm⁻¹ while the broad absorption at 20830 cm⁻¹ is assigned to second transition while the third band is obscured by the intense LMCT charge transfer band.

The room temperature solid-state ESR spectrum of the complex exhibits a broad band centered on g=2.10. At 77° K, however, a sharp signal is found at g_{Ô}=2.253 with a shoulder around g_{Ô}=2.058 suggesting a distorted square planar geometry around the copper center. Moreover, the copper hyperfine structure is also observed on g_{Ô} component of the spectrum with a characteristic value of 142×10⁻⁴ cm⁻¹ suggestive of planar structure. The distortion factor, f(a), calculated for this compound (159 cm⁻¹), is indicative of moderate distortion in the square planar geometry. It is plausible that the distortion may be caused due to in-equivalent bonding of thiosemicarbazone and halide ligands inducing deviation of metal conjugate.

### Molecular Modeling Studies.

Docking experiments were conducted on FPA-101with the MDS Suite software (MDS 1.0 Molecular Design Suite, available from Vlife Sciences Technologies, Pvt Ltd Pune, India, (2003)) using the published crystal structure of estradiol bound with the estrogen receptor, ERα. The protein was energy minimized after removing the ligand and adding the hydrogens using MMFF94 force field until the gradient reached 0.1 kcal/mol-A. The energy minimized structure was then utilized for the study of interactions of several conformers of FPA-101. The conformers with the best binding energies were chosen for observation of the interaction of the ligand with the key residues in active pocket of the ER-α.

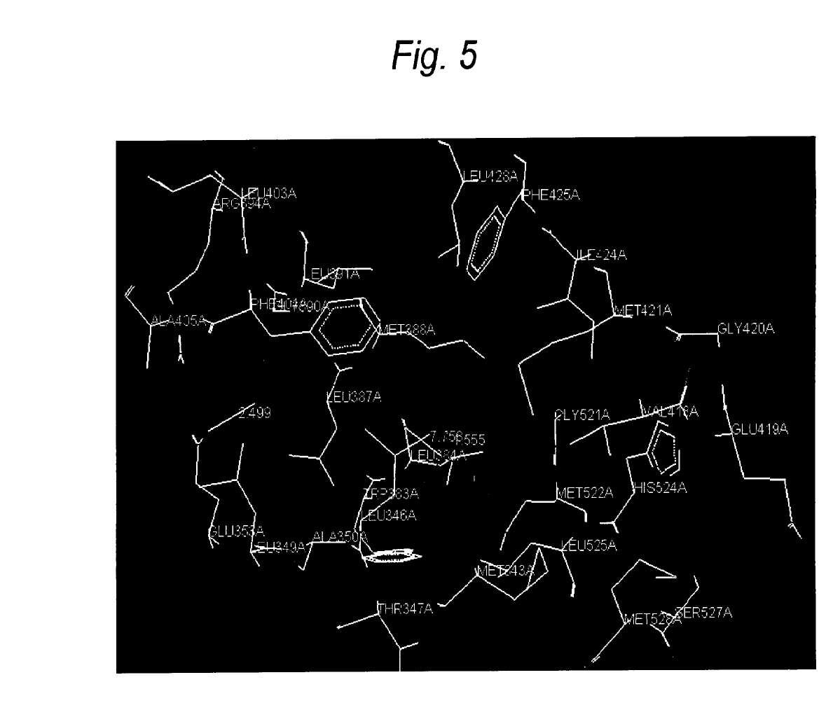

In addition to the foregoing, the binding energies were also calculated to determine the affinity of the ligand as compared to the agonist, estradiol. It was observed that FPA-101 can enter the protein cavity of ER-α without disturbing the protein at the level of HIS 524 while the side chain in it was primarily responsible for the antagonistic effect as it anchored onto the protein residues with a slight bend as shown in FIG. 5. The binding energy calculated for FPA-101 was found to be 20.34 kCal/mol indicating that FPA-101 has better binding with ER which probably is responsible for the potent cell growth inhibitory action observed in vitro assays reported hereinbelow.

The anchoring of the side chain appears to involve an extensive hydrogen bonding network with GLU 353 and PHE 404 of the main chain as well as some pi interactions with PHE 404. Stabilization of the compound FPA-101 within the ligand binding domain was also promoted through hydrophobic and van der Waals interactions of the side chain substituents with several protein residues such as MET343, LEU346, LEU349, ALA 350, LEU 384, LEU387, LEU 391, ARG394, VAL 418, MET241, 1LE424, GLY521, LEU525 and MET528, respectively.

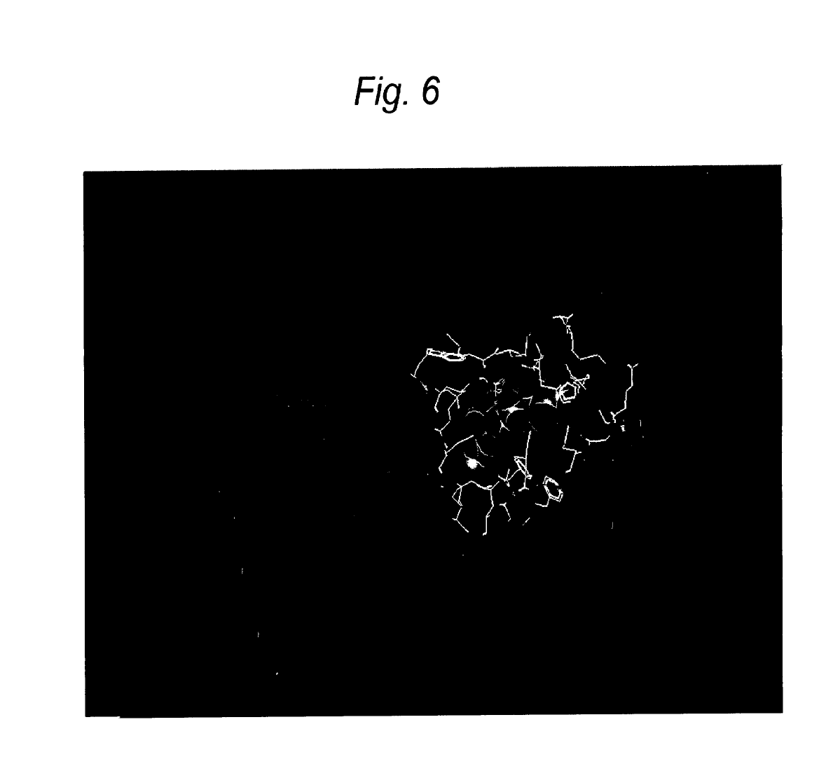

Referring to FIG. 6, the superimposed docking images of estradiol and the Schiff base FPA-101 with ER-α indicates that the side chain in FPA-101 modifies the position of the helix 12 of the estrogen receptor in such a manner as to inhibit co-activator recruitment resulting in enhanced anti-proliferative activity.

### In Vitro Cell Growth and Proliferation Assays

The compounds were tested in multiple panels of breast (MCF-7, T47D, BT20 and MDA-MB231), prostate (PC3), and pancreatic (BxPC3, COLO357) cancer cell lines, using the dye 3-[4,5 dimethylthiazol-2-yl]-2,5 diphenyltetrazolium bromide (MTT assay) according to a method described in Banerjee, et al., Cancer Research, Vol. 62, page 4945 (2002).

More specifically, the tumor cell lines MCF-7, MDA-MB 231 and COLO 357 were maintained in Dulbecco's modifed Eagle's medium (DMEM) with phenol red. Cell lines T47D, BT20, PC3 and BxPC3 were routinely cultured in RPMI medium, each medium being supplemented with Penicillin (50 U/ml), Streptomycin (50 μg/ml) and 10% fetal calf serum (FCS) (Gibco, Myclone). The cell lines were cultured in a humidified incubator in an atmosphere of 5% CO2 and were maintained in continuous exponential growth by twice a week passage.

Adherent cells at a logarithmic phase were detached by addition of 2-3 ml of trypsin (Gibco), 0.02% EDTA mixture and incubation at 37° C. Cells were plated (100 μL per well) in 48 well flat bottom microtiter plates at densities of 2000 to 5000 per cm² (MCF-7, MDA-MB321, T47D, BT20, PC3, BxPC3, COLO357) cells per well. Cells were incubated for 24 h at 37° C. to resume exponential growth. The test compounds were dissolved in DMSO (final concentration 0.1%) and added to the cells 24 h after seeding at varying concentrations. Control wells containing the culture medium, were included in each experiment. Cell proliferation was evaluated after 72h by means of MTT assay.

The IC₅₀ of the Schiff base ligand FPA-101 and its metal conjugates FPA-102 to FPA-104 is summarized in the Table 1. The parent ligand FPA-101 showed moderate antiproliferative action against the cell lines tested and showed high cell kill with low IC₅₀ in pancreatic cell line BxPC3. Highest cell growth inhibition was obtained for the copper conjugate FPA-102 with IC₅₀ values in the range 5-13 μM as shown in Table 1.

TABLE 1

| | | | | | | | | Fluorescence |
|---|---|---|---|---|---|---|---|---|
| | MTT cell viability assay IC₅₀ (μM) | | | | | | | polarization |
| | | MDA- | | | | | | assay IC₅₀ |
| Cmpd | MCF-7 | MB 231 | T47D | BT20 | PC3 | BxPC3 | COLO357 | (μM) |
| FPA-101 | 20 | 25 | 15 | 53 | 16 | 5 | 13 | |
| FPA-102 | 6 | 11 | 5 | 13 | 9 | 12.7 | 9 | 15 |
| FPA-103 | 7.5 | 8 | NE | >100 | >100 | ND | 20 | |
| FPA-104 | 22 | 34 | NE | 18 | 18 | 5 | 7.3 | |

### Apoptosis Assays

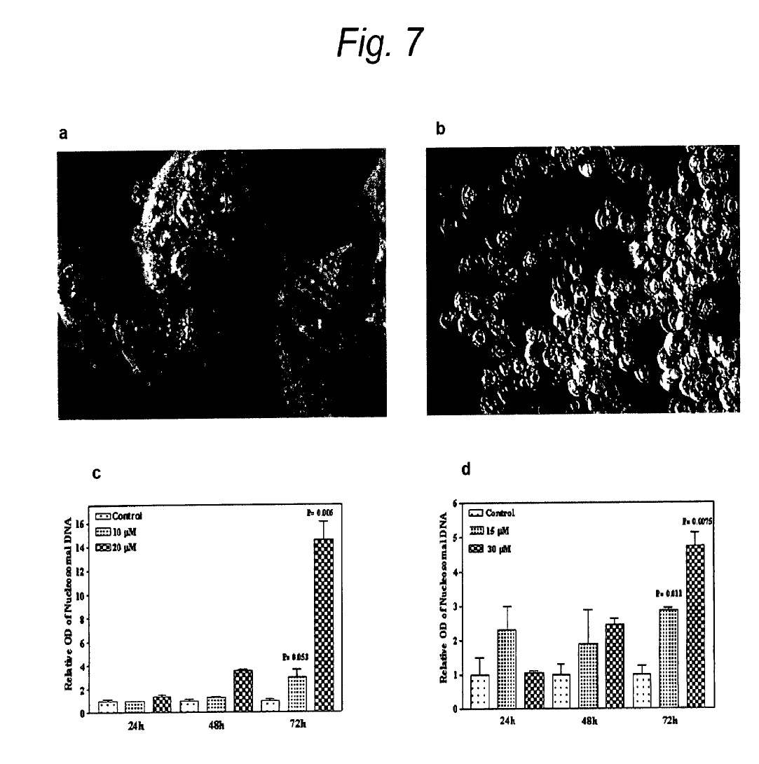

To determine whether the metal conjugates induce apoptosis in tumor cells, quantitative evaluation of apoptosis was carried out with a Cell Death Detection ELISA kit (Roche, Palo Alto, Calif.). Apoptosis was detected according to the protocol supplied by the manufacturer. Briefly, BT20, PC-3, BxPC3, and Colo357 cells were treated with two concentrations of the metal conjugates FPA-102 to FPA-104, specifically at IC₅₀ (10 μM) and above IC₅₀ (20 μM), and then subjected to apoptosis assay. The results for the copper conjugate FPA-102 in two cell lines BT20 and PC3 are shown in FIGS. 7(c) and 7(d).

To provide further evidence of apoptosis, we carried out phase contrast microscopy using a mixture of propidium iodide (PI) and Hoechst dye 33342 to determine the integrity of cell membranes of cancer cells. The quantification of the apoptosis was carried out by cell count after staining with PI followed by visualization of treated cells by fluorescence analysis. The fluorescent dyes PI and Hoechst 33342 were added to the cell culture medium after treatment with the test compounds (10 μM) for 48 h at a final concentrations of 10 μg/ml (PI dye) and Hoechst 33342 (1 μg/ml), respectively. After 5 minutes at 37° C. the cells were placed under a fluorescence microscope (Nikon Optiphot) with a filter block giving excitation at 380 nm and 480 nm.

As shown in FIG. 7b, cells with disrupted membranes preferentially gave strong red nuclear fluorescence due to the uptake of PI whereas cells with intact cell membranes gave blue nuclear fluorescence, due to uptake of the cell permeable Hoechst 33342 fluorochrome. Referring to FIG. 7b, the cells treated with IC₅₀ concentrations of the copper conjugate showed nuclear condensation and membrane damage characteristic of cells undergoing apoptosis as compared to the control cells. This suggests that the choice of the metal for the conjugate is very important and should be dictated by its hard-soft nature and redox behavior. Among the present series of metal conjugates, the copper complex is the redox active compound which exhibits the highest potency of inducing apoptosis indicating that redox cycling may indeed be an important contributing factor to the antiproliferative activity of these compounds.

Other results indicate that after 3 h of incubation with the copper conjugate FPA-102 at IC₅₀ concentration, 40% of the cells were non-viable while nearly 95% of the cells were apoptotic after 24-48 h treatment (data not shown).

### Fluorescence Polarization Assay and Western Blot Assay

We had previously shown that the parent genistein shows inhibition of Akt kinase (Li, et al., Clin. Can. Res., Vol. 8, page 2369 (2002)). Therefore, the present metal conjugates were tested by using fluorescence polarization-based assays for detection of serine/threonine kinase activity.

The serine threonine Akt kinase reaction was carried out in 96 well black plates in a total volume of 100 μl using Ser/Thr kinase polarization based assay kit from Invitrogen Part #P3103. Briefly, the Akt kinase assay reaction was set up in a total volume of 40 μl in the presence of different concentrations of inhibitor. Stock solutions of test compounds were made in DMSO. From DMSO stocks, 4× solutions of test compounds were prepared in 1× assay dilution buffer; 10 μl of this drug solution was used in the assay. Each reaction well had 25 ng of Akt1/PKBα, active enzyme (Upstate Biotechnology, Charlottesville, Va., Cat #14-276), 0.4 mM GSK-3 peptide, (custom synthesized), ATP (100 μM) from Sigma Chemicals, St. Louis, Mo., Cat #A5394, and the test compound to be tested. The plate was incubated at 30° C. for 30 minutes. Enzyme reaction was quenched with 10 μl of 50mM EDTA for 5 minutes. Anti phosphor serine antibody was added 25 μl/well followed by 10 μl of fluorescent tracer. The total volume was made up to 100 μl by adding 15 μl of fluorescence polarization assay dilution buffer. Appropriate controls and blanks were prepared. The plate was incubated for 1 h at RT and was analyzed for polarization at 435 excitation and 535 emission wavelength on Tecan Ultra microplate reader. Polarization values were normalized to the control and were plotted against log concentration of test compounds using Graph Pad Prism software. Sigmoidal dose response curve analysis was used to calculate the IC₅₀ of test compounds.

It was observed that FPA-102 inhibits Akt kinase activity at 15 μM, as shown in Table 1, whereas the parent ligand (FPA-101) and the nickel and platinum conjugates (FPA-103 and FPA-104, respectively) required a higher concentration for kinase inhibition (data not shown).

The status of Akt in PC3 cells treated with FPA-102 after 72 hours was studied by Western blot assay.

PC3 cells were plated on culture dishes and allowed to grow for 24 h followed by addition of FPA-102 at concentrations of 10 and 20 μM for 72 h. Control cells were incubated in the medium with Na₂CO₃ using same time point. After incubation the cells were lysed with This-HCl (62.5 mm) and 2% SDS. Protein concentration was then measured using BSA protein assay (Pierce Chemical Co., Rockford, Ill.). Cell extracts were subjected to 10% SDS-PAGE and electrophoretically transferred to nitrocellulose membrane. Membranes were then incubated with monoclonal anti-Akt (epitome: amino acids 345-480 of Akt1, 1:500; Ontogeny, 26. San Diego, Calif.), anti-phosphor-Akt Ser473 (1:1000; Cell Signaling, Beverly, Mass.), primary monoclonal anti-PARP antibody (1:5000; Biomol, Plymouth Meeting, Pa.) and anti-β-actin (1:5000; Sigma) antibodies, washed with TTBS, and incubated with secondary antibody conjugated with peroxidase. The signal was then detected using the chemiluminescent detection system (Pierce, Rockford, Ill.).

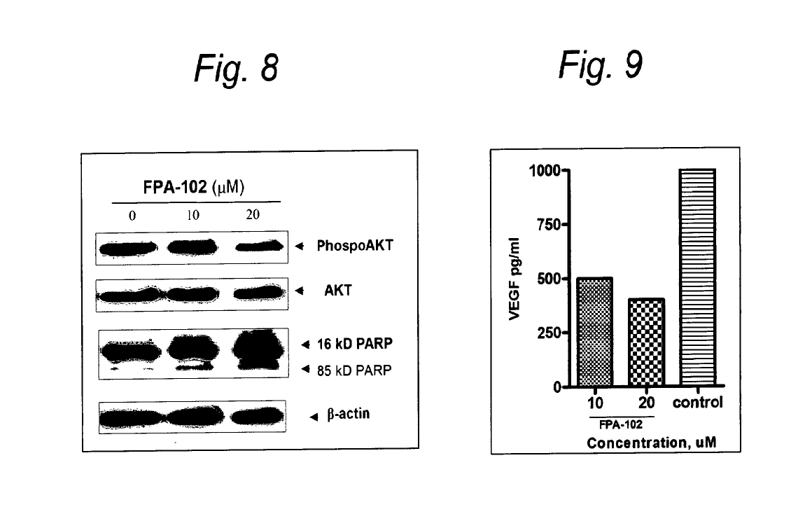

FIG. 8 is the Western blot of Akt, p-Akt and PARP in PC3 cells treated with FPA-102 for 72 hours. FPA-102 down regulates the phosphor-Akt in PC3 cells as seen in FIG. 8. FPA-102 also induces poly (ADP-ribose) polymerase degradation and produces PARP p85 cleaved products indicating apoptotic cell death processes induced by FPA-102.

### In Vitro Expression of VEGF.

The effect of FPA-102 on vascular endothelial growth factor (VEGF), a key angiogenic protein in cancer cells, was investigated in BxPC3 (pancreatic) cancer cells by using the Quntikine human VEGF ELISA kit (R&D Systems, Minneapolis, Minn.). BxPC3 cells (0.1×106/well) were plated into 6-well plates and incubated in supplemented RPMI containing 10% FBS. After 72 hour of incubation with FPA-102 (10 μM, 20 μM), the conditioned medium was aspirated and the VEGF protein levels of the culture medium were determined.

FIG. 9 is a graphical representation of VEGF levels in pg/mg after 72 hours of exposure to FPA-102 at several concentration levels. The copper conjugate causes decrease in the VEGF levels in BxPC3 cells after 72 h treatment as shown in FIG. 9, suggesting that inactivation of AKt by FPA-102 down regulates NF-κB and decreases expression of NF-κB.

### Animal Studies

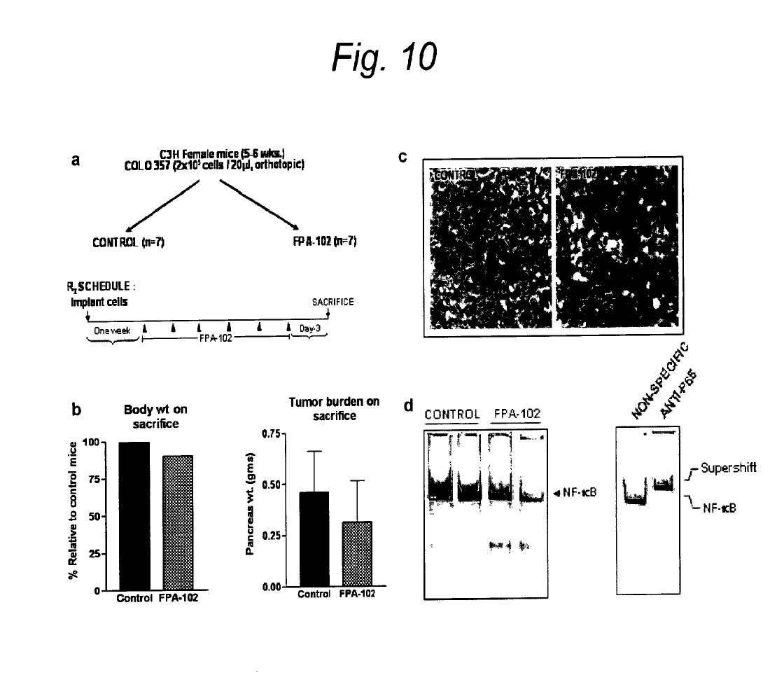

The therapeutic efficacy of copper conjugate FPA-102 was evaluated (i.v injection on alternate days for 6 days) in an established orthotopic pancreatic animal model using COLO 357 cells. As shown in as in FIG. 10a, FPA-102 was able to decrease tumor burden without causing any toxicity as reflected by no significant change in the body weight of treated mice as compared to control (see, FIG. 10b).

The redox sensitive transcription factor NF-κB, is downstream of the Akt kinase protein and plays major role in the cell proliferation and metastasis and resistance in major cancers such as breast, prostate and pancreatic. In order to determine the effect of FPA-102 on this downstream transcription factor in the orthotopically growing pancreatic tumor model, we performed an Electrophoretic Mobility Shift Assay (EMSA) by incubating 8 μg of nuclear protein with IR Dye TM-700 labeled NF-κB oligonucleotide. Nuclear proteins were extracted from the tumor tissue as described previously (see, for example, Banerjee, et al., Cancer Research, Vo. 65, p. 9064 (2005)) and kept at −70° C. until use. Protein concentration was determined using the bicinchonic acid assay kit with BSA as the standard (Pierce Chemical Co., Rockford, Ill.). The incubation mixture included 25 mM DTT and 2.5% Tween 20 in a binding buffer. The DNA-protein complex formed was separated from free oligonucleotide on 8.0% native polyacralyamide gel using buffer containing 50 mM Tris, 200 mm glycine, pH 8.5, and 1 mM EDTA, and then visualized by Odyssey Infrared Imaging System using Odyssey Software Release 1.1.

FPA-102 causes down-regulation of NF-κB in the tumor tissue after treatment for 10 days with FPA-102 once every alternate day as shown in FIG. 10d. Tumor histology, from both groups, showed high grade carcinoma associated with tumor apoptosis necrosis, and fibrosis. However, there were significant differences in the pattern of necrosis, inflammatory response and fibrosis among these two groups. The control tumor shows small islands of necrosis which comprised less than 20% of the mass. The viable tumor cells form large nests or sheets with minimal stromal fibrosis and inflammatory infiltrates. In contrast, the tumor from treated mice showed marked necrosis and apoptosis. Only 20% of the mass was viable. The viable neoplastic cells form small nests of clusters associated with extensive stromal fibrosis and inflammatory infiltrates (See, FIG. 10c).

In conclusion, the isoflavonoid analogs utilizing the steroidal motif of genistein, and their metal conjugates, can be prepared in a minimum number of steps with good purity and yield. These compounds have been demonstrated to be effective against breast, prostate, and pancreatic cancers.

### II. Non-Steroidal Embodiment

Synthesis of 3-formylchromone Schiff Bases (FPA-120 to FPA-127)

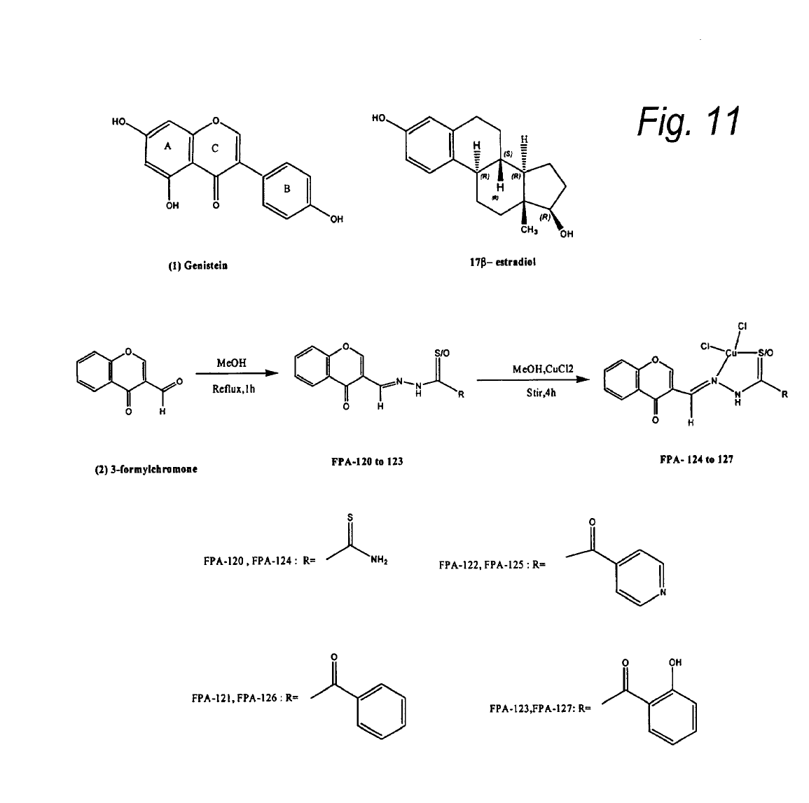

FIG. 11 is a schematic representation of a chemical reaction scheme for synthesizing a non-steroidal embodiment of a pharmacologic agent in accordance with the invention, and its metal conjugate. In the specific embodiment discussed herein, the thiosemicarbazone and hydrazone analogs of 4-oxo-4H-chromene-3-carboxaldehyde (herein designated as chromone) and their Cu(II) complexes are synthesized

Synthesis of Benzoyl Hydrazide

Benzoyl hydrazide was prepared according to a literature method (Furniss, et al., Vogel's Text-book of Practical Organic Chemistry, 5^{th} edition, ELBS: Longman, UK, page 1269 (1989) by refluxing, on a water bath, a mixture of methyl benzoate and hydrazine monohydrate in a 1:1 molar ratio for three hours. The solvent was stripped off on a rotavapor and the solid obtained was re-crystallized from aqueous ethanol and finally dried in a vacuum over anhydrous CaCl₂.

Thiosemicarbazide hydrochloride was prepared as described above. Isonicotinoyl hydrazide and salicylic hydrazide can be prepared according to the methods published in Vogel's Text-book of Practical Organic Chemistry, id., or purchased commercially, illustratively from Sigma Chemical, St. Louis, Mo. (Cat. #13377 and #238848, respectively)

Synthesis of Schiff Base

The Schiff base ligands FPA-120 to FPA-123 were synthesized by mixing equimolar amounts of chromone with various amines in methanolic solvent and maintaining the reaction mixture at reflux temperature for lhr. The products obtained were filtered off, recrystallized from (1:1) DMF-methanol and finally dried in vacuum desiccator over anhydrous CaCl₂. Pale yellow crystals of the Schiff base ligand, suitable for single crystal X-ray diffraction studies, were grown from (1:1) DMF-methanol by slow evaporation.

The selected amines, in the examples reported herein, thiosemicarbazide hydrochloride, benzoyl hydrazide, isonicotinoyl hydrazide, and salicylic hydrazide, respectively, are effective pharmacophores found in many therapeutic compounds currently used in the clinical practice and serve as spacers in the present design, keeping the cytotoxic metal conjugates away from the isoflavonoid moiety. This strategy has been found to be useful for retaining pharmacological properties of both the carrier and cytotoxic moieties.

The following compounds were synthesized:

4-oxo-4H-chromene-3-carboxaldehyde-thiosemicarbazone (FPA-120).

(C17H10N2O3): C, 53.88%; H, 3.32%; N 16.50%; S, 12.76%; (calculated C, 53.44% H, 3.64% N, 17.00% S, 12.95%)

4-oxo-4H-chromene-3-carboxaldehyde-benzoylhydrazone (FPA-121).

(C16H9N3O3): C, 69.86%; H, 4.10%; N 9.58%; (calculated C, 63.43% H, 4.38% N, 9.01%)

4-oxo-4H-chromene-3-carboxaldehyde-isonicotinylhydrazone (FPA-122).

(C16H9N3O3): C, 65.00%; H, 3.48%; N 14.06%; (calculated C, 65.00% H, 3.75% N, 14.33%)

4-oxo-4H-chromene-3-carboxaldehyde-salicylichydrazone (FPA-123). (C17H11N204): C, 65.68%; H, 3.89%; N 9.03%; (calculated C, 66.23% H, 3.87% N, 9.09%)

### Synthesis of Metal Complexes (FPA-124-127)

The copper (II) complexes (Compounds FPA-124 to FPA-127) were synthesized by mixing equimolar amounts of the ligands and CuCl₂×2H₂O in methanol with a trace amount of dimethylformamide. The resulting mixture was refluxed at room temperature for an hour. The precipitates formed were removed by filtration, washed with the methanol solvent and dried in a vacuum over anhydrous CaCl₂.

### Spectroscopy

The ¹H NMR spectra of the ligands FPA-120 to FPA-123 were recorded in d6-DMSO on a Varian-Mercury 300 MHz spectrometer using Si(CH₃)₄ as an internal standard. The ¹H NMR spectra of the ligands FPA-120 to FPA-123 in d6-DMSO exhibits signal in the region 11-12 ppm, which can be attributed to the amide proton and confirms E-isomeric form. The presence of a downfield NH proton for them may be due to the involvement of this group in the hydrogen bonding with d6-DMSO, which is well known for the amide proton. The downfield shift of the —OH proton in FPA-123 that resonates at 11.80 ppm indicates that the —OH proton in this ligand is probably involved in the formation of strong intra-molecular hydrogen bonding. Thus, ¹H NMR spectra of the ligands FPA-120 to FPA-123 displaying signals corresponding to the —NH and —OH protons confirms existence of the keto form and absence of deprotonation in these compounds. The aromatic protons appear in the range 6-8.2 ppm for all compounds.

An ORTEP drawing and unit cell packing diagram of the X-ray crystal structure of ligand FPA-120 (not shown), and the bond distances and bond angels, demonstrated that the pharmacophoric side chain is co-planar with the chromone moiety while thiocarbonyl sulphur and the azomethine nitrogen atoms are placed in the trans positions with respect to the hydrazinic bond corresponding to the E isomer. The molecular conformation is stabilized by the presence of strong intermolecular hydrogen bonds between N (2)-H (2) . . . O (2) (2.09 Å) and N (3)-H (32) . . . S (1) (2.49 Å) linkages, respectively, which is a common feature in many thiosemicarbazonate compounds. This compound contains an isothiocyanate function (although not terminal as in sulphoraphanes) which has been shown to be capable of activating MAPKs, NRF, ARE-mediated luciferase reporter genes and phase II enzyme gene induction.

The spectroscopic data indicates that during metal conjugation, ligands FPA-120 to FPA-123 behave as bidentate thionic moieties coordinating through azomethine nitrogen and thiocarbonyl sulphur/enolic hydroxyl (in case of hydrazonates), respectively. The electronic spectra confirm the square planar geometries for the copper conjugates with presence of 2B1g→2A1g and 2B1g→2Eg transitions, respectively. The absorption at 25000 cm⁻¹, observed for the compound FPA-124, is ascribed to S→Cu (II) charge transfer band, while the absorption in the region 22000-24000 cm⁻¹ is due to oxygen to copper charge transfer transition. The magnetic moments of the copper compounds (1.74-1.94 BM) are typical of monomeric compounds having distorted square planar geometries. The EPR parameters further confirm such geometries with a parametric relation gll>g⊥>2.0 and All values around 175-140 gauss. The distortion factor f (given by gll/All) calculated for the present compounds are comparable with analogous copper complexes reported in the literature (Sonawane, et al., Polyhedron, Vol. 13, page 395 (1994). This parameter may be important for designing copper compounds having radical scavenging properties.

The electrochemical profile of Compound FPA-120 has an irreversible reduction peak centered at −1.30 V which is due to the reduction of azomethine (C═N) function. A similar peak is also observed in case of compounds FPA-121 to FPA-123. An additional quasi-reversible peak at −0.65 V is assigned to the reduction of the aroylhydrazone moiety in case of the hydrazonate ligands. All copper complexes show a reversible Cu⁺²/Cu⁺¹ redox couple in the range +0.28 to +0.35 V indicating a facile reduction of the cupric center. The analytical data, crystal data, bond length and angles, hydrogen bond geometries, and NMR data for Compounds FPA-120 through FPA-127 described herein can be found in tabular form in Barve, et al., Metalloflavonoids as anticancer agents against estrogen independent breast and androgen independent prostate cancers: Synthesis, X-ray crystal structure, spectroscopy, magnetism, electrochemistry and in vitro anticancer activity, J. Medicinal Chemistry, June 2006, the text of which is incorporated herein by reference.

### Molecular Modeling Studies

To investigate the interactions of the parent genistein and its metal conjugates with the active domains of the PKB (Akt) protein, namely the Pleckstrin homology (PH) and the catalytic Kinase domain, molecular modeling was carried out using a structure available in a protein databank (PDB) to represent the Pleckstrin homology domain of human Protein Kinase B α isoform using MDS 2.0 Molecular Design Suite software, available from Vlife Sciences Technologies, Pvt Ltd Pune, India.

Previously, 3′,4′-O-substituted derivatives of isoflavones were developed as therapeutic agents against protein tyrosine kinase (PTK) and molecular modeling studies confirmed that isoflavonoids have the structural features important for binding to the SH2 domain of p56lck protein kinase (West, et al., Structure and Bonding, Vol. 76, page 1, (1991). However, the X-ray crystal structure for the Kinase domain of PKB α isoform was not available in the protein databank. Therefore, the Kinase domain of PKB α was modeled by using the cleaned and optimized X-ray crystal structure of PKB β (PDB Code: 1GZK) as a template [sequence identity (86%) and similarity (94%) with the α-isoform]. The conformation analysis of genistein and its Cu⁺² conjugates FPA-124 to FPA-127 was carried out and conformers satisfying steric requirement of the each cavity were considered for docking. The conformers were placed in the active site and resultant complex structures were then energy minimized using MMFF94 force-field.

The Kinase domain cavity of PKB (Akt) protein is comprised of a buried hydrophobic interior and a relatively less buried hydrophilic surface. While the polar exposed portion of the cavity is characterized by the presence of charged residues such as Glutamate (GLU274, GLU310), Aspartate (ASP270, ASP288) and THR156, the buried hydrophobic interior of the cavity is characterized by presence of hydrophobic residues such as PHE289, LEU291, LEU152, VAL160 and ALA173. Unlike the kinase domain, the PH domain ligand binding cavity is primarily shallow and hydrophilic in nature. The presence of an ample number of Lysine and Arginine residues, such as ARG25, ARG23, LYS14, ARG86, ARG15 and LYS39, renders a positively charged nature to the cavity surface. However, one negatively charged residue, GLU17, is also present on the cavity surface thereby conferring an acidic nature to a relatively smaller region of the cavity surface. It is worth noticing that PH domain cavity lacks a hydrophobic buried interior as observed in the kinase domain. Hence, we believe that these fundamental differences, that is electrostatics and shape-size characteristics of the kinase and PH domain, form the underlying basis for variable binding affinity as well as specificity of different ligands docked into these two domains.

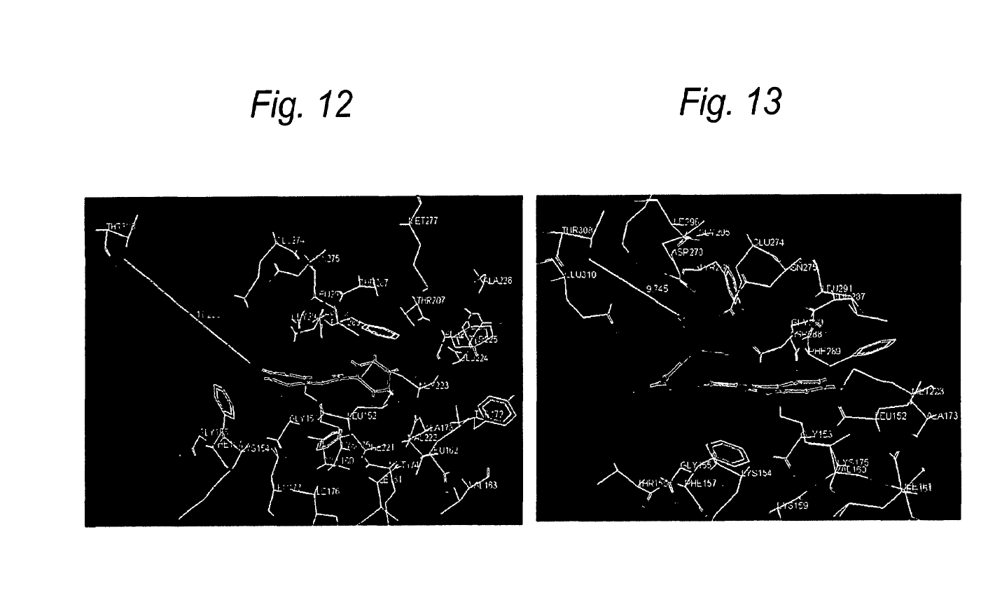

FIG. 12 is a computer-generated model of genistein docked into the kinase domain of PKB (Akt) protein. FIG. 12 shows interactions within the hydrophobic cavity where the phenolic ring structure of genistein hydrogen bonds with C═O of the MET223 and the —OH group of the B ring binds to a carboxyl group on GLU274 in the kinase domain of PKB protein. FIG. 13 is a computer-generated model of FPA-124 interacting with the active site of the kinase domain of PKB protein showing stronger charge interactions, as well as hydrogen bonding, with the N-H of GLY290, electrostatic interactions with carboxyl groups of GLU274, GLU310, ASP270 and ASP288 as well as hydrophobic interactions with residues PHE289, LEU (291,152) and VAL160.

It was observed that the aromatic chromone ring of genistein interacts with the amide hydrogen of ARG25 and the phenolic —OH of the B-ring of genistein interacts with the carboxyl group of GLU17 leading to stabilization in the PH domain. Hydrophobic interactions were not found to contribute to genistein binding in any manner because of lack of any significant hydrophobic residues in the PH domain cavity. Unlike genistein, binding of the metal conjugate FPA-124 in the PH domain was stabilized by additional hydrogen bonding with key amino acids, ARG25, GLU17 AND ILE19, which are involved in the stabilization of the receptor-ligand complex. Since different PH domains normally share 20% sequence identity, this may facilitate the development of drugs that bind specifically to the PH domain of PKB protein. In the present case, it was observed that some charged residues, such as ARG86, ASN53 and LYS 14, were as yet unexploited. This provides an ideal opportunity to further modify, or add substituents to, the benzopyran motif, to yield even more potent molecules with enhanced PKB inhibitory activity.

The crystal structure of the kinase domain of PKB protein bound to 5′-adenyl-amido-diphosphate (AMP-PNP) is an inhibitor analogous to the natural substrate ATP as reported in the literature (PDB ID: 1O6K). The AMP-PNP ligand does not directly bind to THR308 and yet prevents its phosphorylation. It was seen that the genistein molecule interacts with the hydrophobic cavity of the Kinase domain of PKB protein where its single phenolic ring structure orients itself to hydrogen bond with the C═O of the MET223, and the hydroxyl group of B ring binds to carboxyl group of GLU274 (see, FIG. 12). The docking studies of conjugate FPA-124 indicate that the metal conjugate has three kinds of interactions with the kinase protein. The hydrophobic chromone end of FPA-124 interacts with the hydrophobic interior of the cavity, namely the hydrophobic side-chains of the residues PHE289, LEU (291,152) and VAL160. Secondly, there exists a hydrogen bond between the backbone N—H of GLY290 and the ethereal oxygen atom of FPA-124. As the copper atom carries a partial positive charge, the metal conjugate interacts with the carboxyl groups of GLU274, GLU310, ASP270 and ASP288 through electrostatic interactions (See, FIG. 13).

It is apparent, from the computer-generated models, that genistein has one more hydrogen bond interaction with the PKB protein than the conjugate FPA-124. Conjugate FPA-124, on the other hand, shows charge interactions within the kinase domain. Hence, it is reasonable to postulate that metal conjugate FPA-124 has better stabilization than the parent genistein because charge interactions are stronger than hydrogen bonding. As a result, metal conjugate FPA-124 has more potent kinase inhibitory activity than genistein. Therefore, the docking models corroborates the fluorescence polarization IC₅₀ data, as reported below in Table 2.

TABLE 2

| | | | | | Fluorescence |
|---|---|---|---|---|---|
| | MTT Cell Proliferation | | | | Polarization |
| | Assay IC₅₀ (μM) | | | | Akt kinase |
| Compound | COLO357 | BxPC3 | BT20 | PC3 | Assay IC₅₀ (μM) |
| Genistein | 50 | 30 | 46-70 | 50 | >70 |
| FPA-124 | 34 | 55 | 7 | 10 | 0.1 |
| FPA-125 | 30 | 20 | 12 | 15 | 15 |
| FPA-127 | 16 | 22 | 12 | 14 | 8 |

### In Vitro Cell Growth and Proliferation Assays

The biological effects of these compounds were studied against the hormone independent breast (BT20) and prostate (PC3) cancers as well as K-ras positive (Colo357) and K-ras negative (BxPC3) pancreatic cancer cell lines. The synthetic pharmacologic agents of the present invention inhibited cell proliferation in all cell lines tested. Moreover, the metal conjugates of the present invention exhibited dose dependent growth inhibitory effects in all of the cell lines.

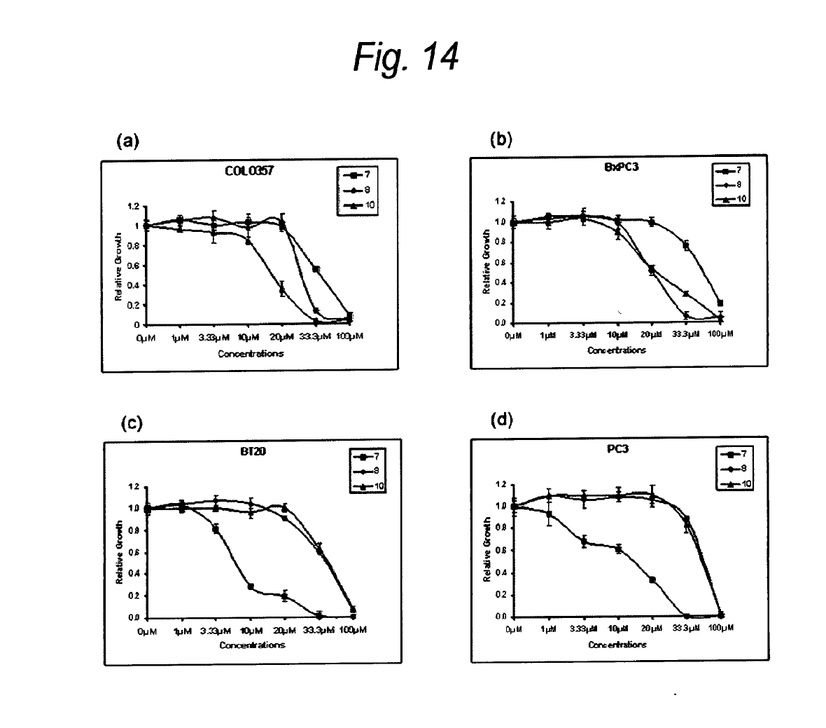

Referring to FIGS. 14(a)-14(d), which are graphical representations, of the inhibitory effects of the metal conjugates FPA-124, FPA-125, and FPA-127 to the relative growth of Colo357, BxPC3, BT20, and PC3 cancer cells, respectively, as a function of concentration (in μM) of the test compounds. Of particular note, compound FPA-124 showed 50% cell kill at 7 μM in BT20 cells and 10 μM in PC3 cells. The metal conjugates FPA-124, FPA-125, and FPA-127 inhibited growth of BxPC3 and COLO 357 cells at concentration >20 μM. The metal conjugate FPA-126 showed cell kill in all the four cell lines at very high doses (>50 μM; data not shown in table or figures). A synergistic enhancement in the anti-proliferative activities was observed upon metal conjugation compared to their corresponding parent ligands (data not shown) as well as to the parent genistein which has IC₅₀ values >40 μM in these multiple cancer cell lines. Metal conjugation confers a pleiotropic characteristic to the organic ligands which is essential for treating a heterogenous disease like cancer. Compounds with well-defined, but singular targets, have been found to have limited therapeutic value due to associated but un-anticipated side effects and rapidly-built resistance.

In addition to pleiotropic effects, metal conjugates are able to assert effects on multi-target sites (whether in intact or dissociated configurations) and, hence, exhibit superior activity and comparatively less frequency of resistance. A comparison of the IC₅₀ values of present metal conjugates with those of genistein revealed substantial decrease indicating therapeutically achievable efficacy (See, Table 2 above).

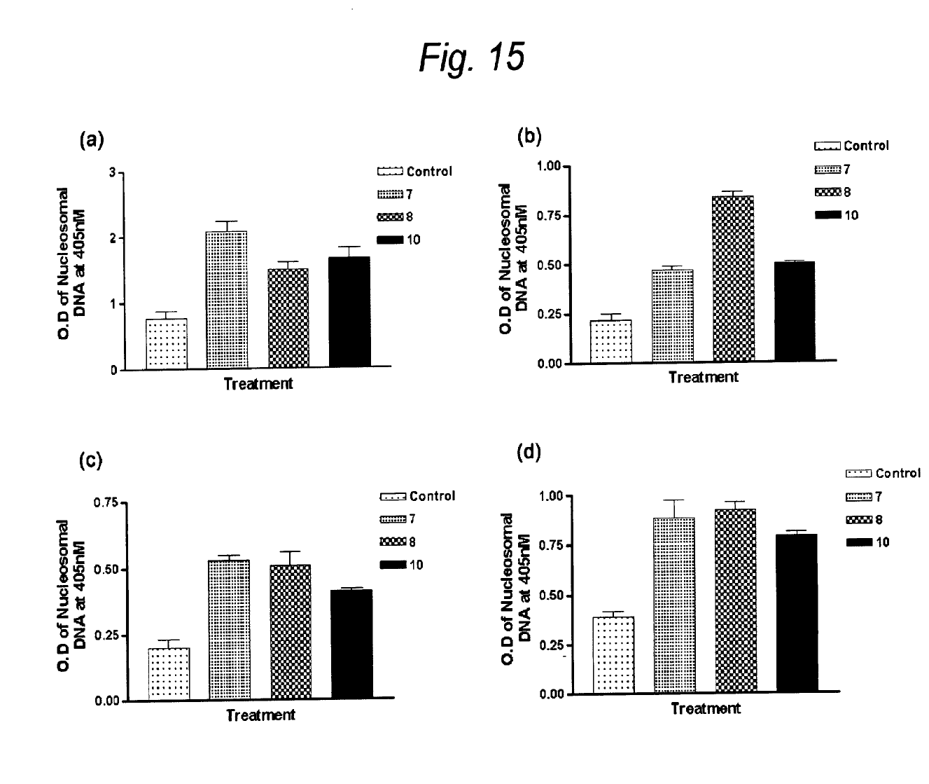

Since all copper compounds are redox active metal conjugates, we believe that redox triggered oxidative stress may be one of the underlying mechanisms for the observed apoptotic cell death in the present case. The quantitative evaluation of the apoptosis by ELISA is shown in FIGS. 15(a) to (d). Referring to FIG. 15, metal conjugate FPA-124 is seen to be the most active compound. This observation is also in accordance with the favorable distortion factor calculated for the present copper compounds using EPR spectra, which factor helps stabilize cuprous and cupric species without dissociation of the conjugate.

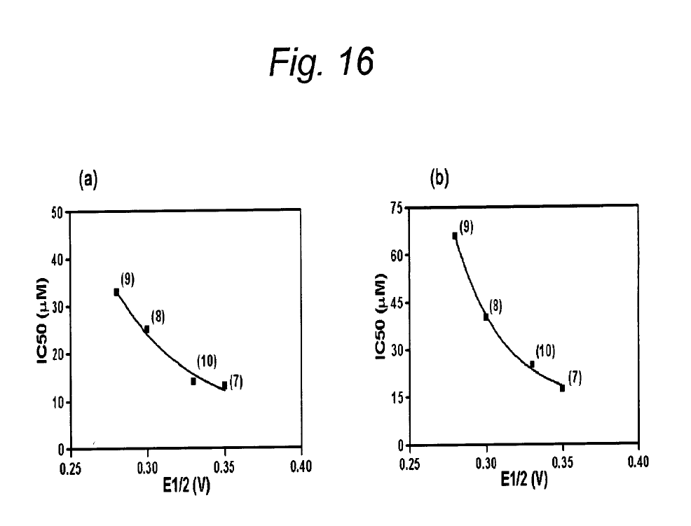

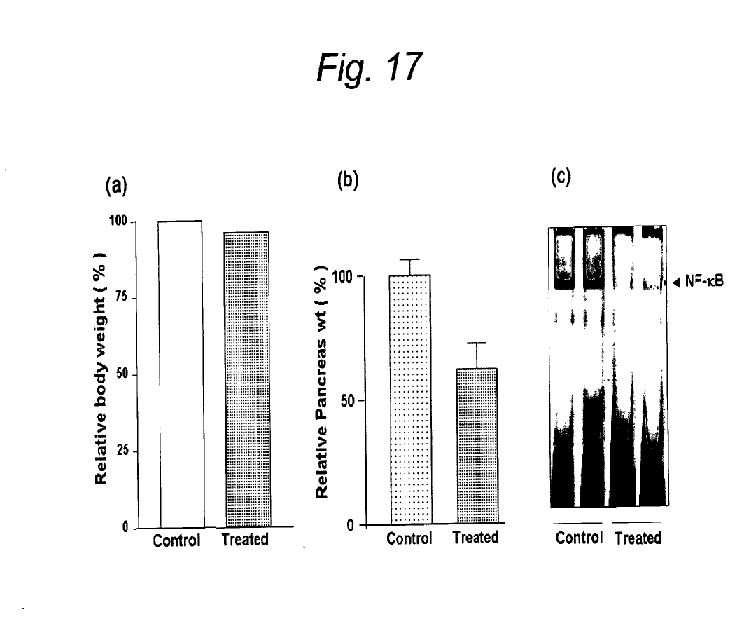

Referring to FIG. 16, which is a graphical representations of the IC₅₀ values (μM) of Compounds FPA-124 to FPA-127 plotted against the metal redox couple, E_{1/2} (V), an inverse relationship was observed between the metal redox couple and the IC₅₀ values for the metal conjugates against the hormone independent breast (FIG. 16a) and prostate cancer (FIG. 16b) cell lines, indicating that this parameter can indeed be used as a guideline for developing effective anti-tumor metal conjugates against these cancers.

In further experiments using fluorescence polarization-based assays, we investigated whether the effects of metal conjugates FPA-124, FPA-125, and FPA-127 could be mediated by inactivation of serine/threonine kinase activity, since PKB (Akt) signaling is important in cancer development inasmuch as it promotes cell survival by inhibiting apoptosis through inactivation of pro-apoptotic factors. The

PKB (Akt) protein has been shown to influence many transcription factors that are involved in controlling the cell growth and survival such as E2F, NF-κB and CREB, and is also known to crosstalk with the RAF/Erk signaling pathways. The PKB (Akt) protein is activated by phospholipid binding and phosphorylation at THR308 by PDK1 or SER473 by PDK2, respectively.

Referring to the IC₅₀ values of these compounds as presented in Table 2, FPA-124 exhibited the lowest IC₅₀ value compared to the other copper conjugates in inhibiting Akt kinase activity. It is interesting to note, however, that while Akt kinase activity could be inhibited with 100 nM (IC₅₀) of FPA-124, the IC₅₀ for inhibiting cell growth is 70-100 fold greater than the inhibition of kinase activity.

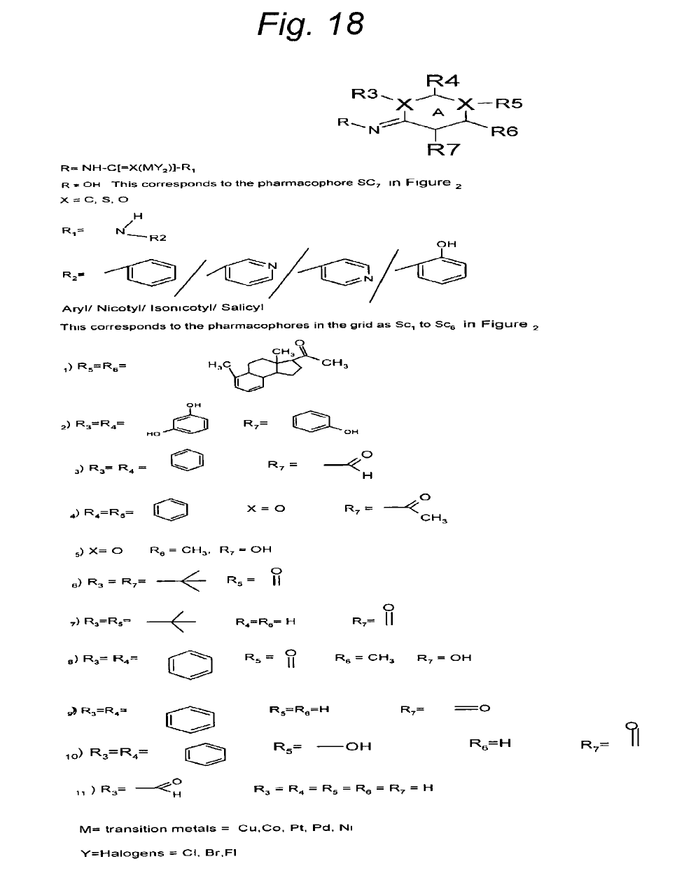

Activation of NF-κB in cancer cells has been shown to attenuate apoptosis induced by chemotherapeutic agents resulting in lower cell-killing and drug resistance. Since the NF-κB pathway is regulated by Akt protein, the effects of the FPA-124 on NF-κB activity was studied in an in vivo experiment, specifically the well-established orthotopic pancreatic tumor model using COLO 357 cells. The parent genistein compound has been shown to inhibit the activity of NF-κB and the growth of hormone dependent (LnCaP) and hormone independent (PC3) human prostate cancer cell lines in vivo without causing systemic toxicity. Recently, we have also observed the potentiating effects of genistein leading to inhibition of tumor growth by radiation in a prostate cancer orthotopic model, and the effect of chemotherapy in orthotopic pancreatic cancer model. In this study, compound FPA-124 had no apparent animal toxicity, as indicated by no change in the body weight of the treated animals (FIG. 17a), but caused a decrease in the tumor load (FIG. 17b). Most importantly, the NF-κB activity was significantly decreased in the tumor tissue of animals treated with FPA-124, supporting the theory that compound FPA-124 produces an in vivo effect through Akt/NF-κB pathway, shown in the EMSA shift assay of FIG. 17c.

In view of the foregoing, it is clear that the novel ligands of the present invention, and their metal conjugates, have the potential to be potent anti-cancer agents. It is to be understood, of course, that while the novel pharmacologic agents are alleged to be useful for the treatment and/or prevention of cancer, the agents may find applicability in the treatment and/or prevention of a variety of other diseases and conditions.

Although the invention has been described in terms of specific embodiments and applications, persons skilled in the art may, in light of this teaching, generate additional embodiments without exceeding the scope or departing from the spirit of the claimed invention. Accordingly, it is to be understood that the drawing and description in this disclosure are proffered to facilitate comprehension of the invention and should not be construed to limit the scope thereof.

What is claimed is:

1. A pharmacologic agent comprising:
a carrier moiety that is a isoflavonoid or isoflavonoid mimetic covalently attached to a cytotoxic pharmacophore to form a ligand.

2. The pharmacologic agent of claim 1 which is a 1:1 complex of a metal ion with the ligand.

3. The pharmacologic agent of claim 1 wherein the carrier moiety is an isoflavonoid mimetic which is a non-fragmented steroidal hormone.

4. The pharmacologic agent of claim 3 wherein the non-fragmented steroidal hormone is selected from the group consisting of estrogen, progesterone, testosterone, and hydrocortisone and prednisone.

5. The pharmacologic agent of claim 4 wherein the non-fragmented steroidal hormone is progesterone.

6. The pharmacologic agent of claim 1 wherein the pharmcophore is selected from the group consisting of amines, alkylamines, arylamines, heterocyclic amines, phenylamines, naphthoylamines, isothiocyanates, semicarbazides, thiosemicarbazides, hydrazones, thiourea, hydroxamates, arylazo, azocylic, carboxyamidrazones, ferrocenes and substituted ferrocenes.

7. The pharmacologic agent of claim 6 wherein the pharmacophore is selected from the group consisting of thiosemicarbazone, benzoyl hydrazide, isonicotinoyl hydrazide, and salicylic hydrazide.

8. The pharmacologic agent of claim 2 wherein the metal ion is a transition metal ion.

9. The pharmacologic agent of claim 8 wherein the metal ion is selected from the group consisting of copper, nickel, and platinum

10. The pharmacologic agent of claim 9 wherein the metal ion is copper.

11. The pharmacologic agent of claim 1 which is 17-acetyl-10,13-dimethyl-1,2,6,7,8,9,11,12,13,14,15,16,17-tetradecahydrocyclopenta[a]phenanthren-3-thiosemicarbazone and its transition metal complex.

12. The pharmacologic agent of claim 1 wherein the carrier moiety is a non-steroidal isoflavonoid or Isoflavonoid mimetic.

13. The pharmacologic agent of claim 12 wherein the non-steroidal isoflavonoid or Isoflavonoid mimetic is an analog of genistein.

14. The pharmacologic agent of claim 13 wherein the analog of genistein is chromone.

15. The pharmacologic agent of claim 14 wherein the pharmacophore is a thiosemicarbazone or hydrazone.

16. The pharmacologic agent of claim 14 which is selected from the group consisting of 4-oxo-4H-chromene-3-carboxaldehyde-thiosemicarbazone, 4-oxo-4H-chromene-3-carboxaldehyde-benzoylhydrazone, 4-oxo-4H-chromene-3-carboxaldehyde-isonicotinoylhydrazone, 4-oxo-4H-chromene-3-carboxaldehyde-salicylhydrazone, and their transition metal complexes.

17. A formulation comprising:
a therapeutically effective amount of a compound according to claim 1; and
a non-toxic delivery vehicle.

18. A method of treating comprising:
administering a therapeutically effective amount of a compound of claim 1, or a pharmaceutically acceptable salt thereof, to a human or animal.

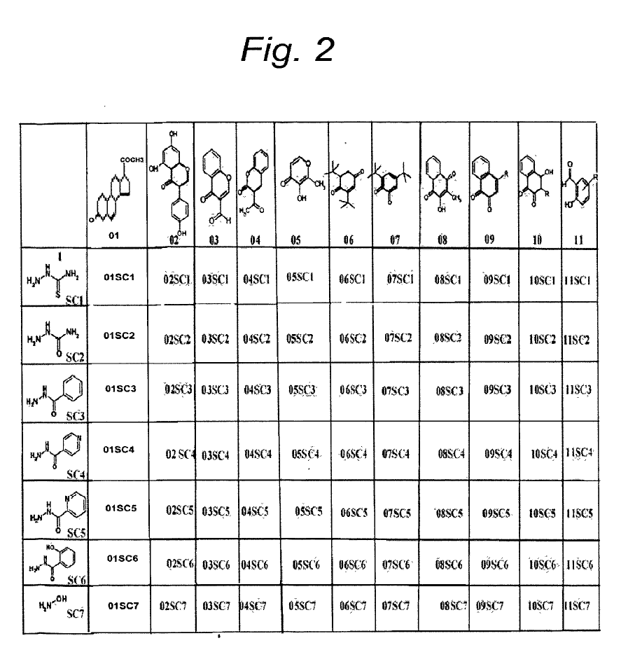

19. A combinatorial library of flavonoid analogs and their metal complexes that can be formed by reacting pharmacophores with privileged core structures derived from the flavonoid or flavonoid mimetics as shown in FIG. 2.
